(19) United States
(12) Patent Application Publication
(10) Pub. No.: US 2002/0165222 A1
(43) Pub. Date: Nov. 7, 2002

(54) Protease inhibitors

(75) Inventors: Robert Wells Marquis JR., Wayne, PA (US); Daniel Frank Veber, Ambler, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/188,165

(22) Filed: Jul. 2, 2002

Related U.S. Application Data
(63) Continuation of application No. 09/913,429, filed on Aug. 14, 2001.
Application No. 09/913,429 is a 371 of international application No. PCT/US00/04133, filed on Feb. 17, 2000.
(60) Provisional application No. 60/120,699, filed on Feb. 19, 1999.

(51) Int. Cl. A61K 31/553; A61K 31/573

(52) U.S. Cl. 514/211.01; 544/162; 514/237.8; 540/544

(57) ABSTRACT

The present invention provides 7-14 membered ring ether protease inhibitors and pharmaceutically acceptable salts, hydrates and solvates thereof which inhibit proteases, including cathepsin K, pharmaceutical compositions of such compounds, novel intermediates of such compounds, and methods for treating diseases of excessive bone loss or cartilage or matrix degradation, including osteoporosis; gingival disease including gingivitis and periodontitis; arthritis, more specifically, osteoarthritis and rheumatoid arthritis; Paget's disease; hypercalcemia of malignancy; and metabolic bone disease, comprising inhibiting said bone loss or excessive cartilage or matrix degradation by administering to a patient in need thereof a compound of the present invention.

### FIELD OF THE INVENTION

This invention relates in general to 7-14 membered ring ether protease inhibitors, particularly such inhibitors of cysteine and serine proteases, more particularly compounds which inhibit cysteine proteases, even more particularly compounds which inhibit cysteine proteases of the papain superfamily, yet more particularly compounds which inhibit cysteine proteases of the cathepsin family, most particularly compounds which inhibit cathepsin K. Such compounds are particularly useful for treating diseases in which cysteine proteases are implicated, especially diseases of excessive bone or cartilage loss, e.g., osteoporosis, periodontitis, and arthritis.

### BACKGROUND OF THE INVENTION

Cathepsins are a family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J. Biol. Chem. 271, 12517-12524; Drake , F. H., et al., (1996) J. Biol. Chem. 271, 12511-12516; Bromme, D., et al., (1996) J. Biol. Chem. 271, 2126-2132.

Cathepsin K has been variously denoted as cathepsin O or cathepsin O2 in the literature. The designation cathepsin K is considered to be the more appropriate one.

Cathepsins function in the normal physiological protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0, 603, 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I collagen represents the major structural protein of bone comprising approximately 90% of the protein matrix. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodelling at discrete foci throughout life. These foci, or remodelling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for a cysteine proteases in bone resorption. For example, Delaisse, et al., Biochem. J., 1980, 192, 365, disclose a series of protease inhibitors in a mouse bone organ culture system and suggest that inhibitors of cysteine proteases (e.g., leupeptin, Z—Phe—Ala—CHN₂) prevent bone resorption, while serine protease inhibitors were ineffective. Delaisse, et al., Biochem. Biophys. Res. Commun., 1984, 125, 441, disclose that E-64 and leupeptin are also effective at preventing bone resorption in vivo, as measured by acute changes in serum calcium in rats on calcium deficient diets. Lerner, et al., J. Bone Min. Res., 1992, 7, 433, disclose that cystatin, an endogenous cysteine protease inhibitor, inhibits PTH stimulated bone resorption in mouse calvariae. Other studies, such as by Delaisse, et al., Bone, 1987, 8, 305, Hill, et al., J. Cell. Biochem., 1994, 56, 118, and Everts, et al., J. Cell. Physiol., 1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K, a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

Several cysteine protease inhibitors are known. Palmer, (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitriles, α-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0,525,420 A1, EP 0,603,873 A1, and EP 0,611,756 A2 describe alkoxymethyl and mercaptomethyl ketones which inhibit the cysteine proteases cathepsins B, H and L. International Patent Application No. PCT/U.S.94/08868 and and European Patent Application No. EP 0,623,592 A1 describe alkoxymethyl and mercaptomethyl ketones which inhibit the cysteine protease IL-1α convertase. Alkoxymethyl and mercaptomethyl ketones have also been described as inhibitors of the serine protease kininogenase (International Patent Application No. PCT/GB91/01479).

Azapeptides which are designed to deliver the azaamino acid to the active site of serine proteases, and which possess a good leaving group, are disclosed by Elmore et al., Biochem. J., 1968, 107, 103, Garker et al., Biochem. J., 1974, 139, 555, Gray et al., Tetrahedron, 1977, 33, 837, Gupton et al., J. Biol. Chem., 1984, 259, 4279, Powers et al., J. Biol. Chem., 1984, 259, 4288, and are known to inhibit serine proteases. In addition, J. Med. Chem., 1992, 35, 4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta, 701, 328).

1,3-diamido-propanones have been described as analgesic agents in U.S. Pat. Nos. 4,749,792 and 4,638,010.

Thus, a structurally diverse variety of cysteine protease inhibitors have been identified. However, these known inhibitors are not considered suitable for use as therapeutic agents in animals, especially humans, because they suffer from various shortcomings. These shortcomings include lack of selectivity, cytotoxicity, poor solubility, and overly rapid plasma clearance. A need therefore exists for methods of treating diseases caused by pathological levels of cysteine proteases, including cathepsins, especially cathepsin K, and for novel inhibitor compounds useful in such methods.

We have now discovered a novel class of 8-14 membered ring ether compounds which are protease inhibitors, most particularly of cathepsin K.

### SUMMARY OF THE INVENTION

An object of the present invention is to provide 7-14 membered ring ether protease inhibitors, particularly such inhibitors of cysteine and serine proteases, more particularly such compounds which inhibit cysteine proteases, even more particularly such compounds which inhibit cysteine proteases of the papain superfamily, yet more particularly such compounds which inhibit cysteine proteases of the cathepsin family, most particularly such compounds which inhibit cathepsin K, and which are useful for treating diseases which may be therapeutically modified by altering the activity of such proteases.

Accordingly, in the first aspect, this invention provides a compound according to Formula I.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides a method of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis.

### DETAILED DESCRIPTION OF THE INVENTION

The present invention provides compounds of Formula I:

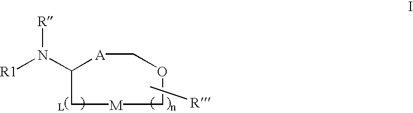

wherein:

A is C(O) or CH(OH);

R¹ is

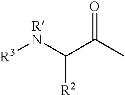

R′ is selected from the group consisting of: H, C₁₋₆alkyl, Ar-C₀₋₆alkyl, and Het-C₀₋₆alkyl;

R″ is selected from the group consisting of: H, C₁₋₆alkyl, Ar-C₀₋₆alkyl, and Het-C₀₋₆alkyl;

R′″ is selected from the group consisting of: H, C₁₋₆alkyl, C₃₋₆cycloalkyl-C₀₋₆alkyl, Ar-C₀₋₆alkyl, and Het-C₀₋₆alkyl;

R² is selected from the group consisting of: H, C₂₋₆alkenyl, C₂₋₆alkynyl, Het, Ar or C₁₋₆alkyl optionally substituted by OR⁶, SR⁶, NR⁶₂, R⁶NC(O)OR⁵, CO₂R⁶, CO₂NR⁶₂, N(C=NH)NH₂, Het and Ar;

R³ is selected from the group consisting of: H, C₁₋₆alkyl, C₃₋₆cycloalkyl-Co₆alkyl, Ar-C₀₋₆alkyl, Het-C₀₋₆alkyl, R⁴C(O)—, R⁴C(S)—, R⁴SO₂-, R⁴OC(O)—, R⁴R⁷NC(O)—, R⁴R⁷NC(S)—, R⁷HNCH(R⁷)C(O)—, and R⁴OC(O)NR⁷CH(R⁷)C(O)—;

R⁴ is selected from the group consisting of: C₁₋₆alkyl, C₃₋₆cycloalkyl-C₀₋₆alkyl, Ar-C₀₋₆alkyl and Het-C₀₋₆alkyl;

R⁵ is selected from the group consisting of: H, C₁₋₆alkyl, Ar-C₀₋₆alkyl, and Het-C₀₋₆alkyl;

R⁶ and R⁷ are selected from the group consisting of: H, C₁₋₆alkyl, Ar-C₀₋₆alkyl, and Het-C₀₋₆alkyl;

M is selected from the group consisting of: HC═CH, H₂C—CH₂; H(OR²)C—C(OR²)H; H(OR²)C—CH₂; H(NR²H)C—C(NR²H)H; H(OR²)C—C(NR²H)H; and H(NR²H)C-CH_{2;}

n is 1-7;

L is 0-1;

or pharmaceutically acceptable salts, hydrates and solvates thereof.

Preferably, A is C(O). Also preferred are compounds according to claim 1 wherein R″ and R′″ are both H. Preferably, n is 1 and L is 0 or 1. Compounds of Formula I wherein M is selected from the group consisting of HC=CH and H₂C—CH₂ are also preferred.

More preferred are compounds of Formula I wherein:

A is C(O);

M is selected from the group consisting of: HC═CH and H₂C—CH₂;

n is 1;

L is 0 or 1; and

R″ and R′″ are both H.

Even more preferred are compounds of Formula I wherein:

A is C(O);

M is selected from the group consisting of: HC═CH and H₂C—CH₂;

n is 1;

L is 1;

R′, R″ and R′″ are independently H;

R² is C₁₋₆alkyl, optionally substituted by OR⁶, SR⁶, NR⁶₂, R⁶NC(O)OR⁵, CO₂R⁶, CO₂NR⁶₂, N(C═NH)NH₂, Het and Ar, preferably isobutyl;

R³ is R⁴C(O)—;

R⁴ is selected from the group consisting of: Ar-C₀₋₆alkyl and Het-C₀₋₆alkyl, preferably:

naphthylenyl, especially naphthylen-2-yl;

benzo[b]thiophenyl, especially benzo[b]thiophen-2-yl;

3-methyl-benzofuranyl, especially 3-methyl-benzofuran-2-yl;

quinoxalinyl, especially, quinoxaline-2-yl;

benzofuranyl, especially benzofuran-2-yl;

benzo[b]thiophenyl, especially benzo[b]thiophene-2-yl;

5-(4-trifluoromethyl-phenyl)-furanyl, especially 5-(4-trifluoromethyl-phenyl)-furan-2-yl;

1-methyl-1H-indolyl, especially 1-methyl-1H-indol-2-yl;

3-methyl-benzofuran, especially 3-methyl-benzofuran-2-yl;

4-methoxy-quinolinyl, especially 4-methoxy-quinoline-2-yl;

5-methyl-benzo[b]thiophenyl, especially 5-methyl-benzo[b]thiophene-2-yl; and

5,6-dimethoxy-benzofuranyl, especially 5,6-dimethoxy-benzofuran-2-yl.

The following compounds of Formula I are particularly preferred embodiments of the present invention:

Naphthylene-2-carboxylic acid[(S)-3-methyl-1-(3-oxo-3,4,5,8-tetrahydro-2H-oxocin-4-ylcarbamoyl)-butyl]-amide;

Benzo[b thiophene-2-carboxylic acid [(S)-3-methyl-]-((S)-3-oxo-3,4,5,8-tetrahydro-2H-oxocin-4-ylcarbamoyl)-butyl]-amide;

3-Methyl-benzofuran-2-carboxylic acid [(S)-3-methyl-1-((S)-3-oxo-3,4,5,8-tetrahydro-2H-oxocin-4-ylcarbamoyl)-butyl]-amide;

Quinoxaline-2-carboxylic acid [(S)-3-methyl-1-((S)-3-oxo-3,4,5,8-tetrahydro-2H-oxocin-4-ylcarbamoyl)-butyl]-amide;

Benzofuran-2-carboxylic acid [(S)-3-methyl-1-((S)-3-oxo-3,4,5,8-tetrahydro-2H-oxocin-4-ylcarbamoyl)-butyl]-amide;

Benzo[b]thiophene-2-carboxylic acid [(S)-3-methyl-1-((S)-3-oxo-oxocan-4-ylcarbamoyl)-butyl]-amide;

5-(4-Trifluoromethyl-phenyl)-furan-2-carboxylic acid[(S)-3-methyl-1-((S)-3-oxo-oxocan-4-ylcarbamoyl)-butyl]-amide;

1-Methyl-1H-indole-2-carboxylic acid [(S)-3-methyl-1-((S)-3-oxo-oxocan-4-ylcarbamoyl)-butyl]-amide;

3-Methyl-benzofuran-2-carboxylic acid [(S)-3-methyl-1-((S)-3-oxo-oxocan-4-ylcarbamoyl)-butyl]-amide;

Quinoxaline -2-carboxylic acid [(S)-3-methyl-1-((S)-3-oxo-oxocan-4-ylcarbamoyl)-butyl]-amide;

4-Methoxy-quinoline-2-carboxylic acid[(S)-3-methyl-1-((S)-3-oxo-oxocan-4-ylcarbamoyl)-butyl]-amide;

5-Methyl-benzo[b]thiophene-2-carboxylic acid [(S)-3-methyl-1-((S)-3-oxo-oxocan-4-ylcarbamoyl)-butyl]-amide;

Benzofuran-2-carboxylic acid [(S)-3-methyl-1-((S)-3-oxo-oxocan-4-ylcarbamoyl)-butyl]-amide;

5,6-Dimethoxy-benzofuran-2-carboxylic acid [(S)-3-methyl-1-((S)-3-oxo-oxepan-4-ylcarbamoyl)-butyl]-amide; and

Benzo[b]thiophene-2-carboxylic acid [(S)-3-methyl-1-((S)-3-oxo-oxocan-4-ylcarbamoyl)-butyl]-amide.

Representative compounds of the present invention are set forth in Examples 1-15.

### Definitions

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L- isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one to five halogens, C₁₋₄alkyl, OR⁶, SR⁶, NR⁶₂, R⁶NC(O)OR⁵, CO₂R⁶, CO₂NR⁶₂, N(C=NH)NH₂, Het and Ar, where R⁵ and R⁶ are defined as herein above. C₀alkyl means that no alkyl group is present in the moiety. Thus, Ar-C₀alkyl is equivalent to Ar.

“C₃₋₆cycloalkyl” as applied herein is meant to include substituted and unsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” means phenyl or naphthyl, optionally substituted by one or more of Ph—C₀₋₆alkyl; Het—C₀₋₆alkyl; C₁₋₆alkoxy; Ph—C₀₋₆alkoxy; Het—C₀₋₆alkoxy; OH, (CH₂)₁₋₆NR⁸R⁹; O(CH₂)₁₋₆NR⁸R⁹; C₁₋₆alkyl, OR¹⁰, N(R¹⁰)₂, SR¹⁰, CF₃, N O₂, CN, CO, R¹⁰, CON(R¹⁰), F, Cl, Br or I; where R⁸ and R⁹ are H, C₁₋₆alkyl, Ph-C₀₋₆alkyl, naphthyl-C₀₋₆alkyl or Het-C₀₋₆alkyl; and R¹⁰ is phenyl, naphthyl, or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quatemized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, C₁₋₆alkyl, OR′, N(R′)₂, SR, CF₃, NO₂, CN, CO₂R′, CON(R), F, Cl, Br and I, where R′ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, thiamorpholinyl sulfoxide, thiamorpholinyl sulfone, and oxadiazoly, as well as triazolyl, thiadiazolyl, oxadiazolyl, isoxazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. EDC refers to N-ethyl-N′(dimethylaminopropyl)-carbodiimide, DMF refers to dimethyl formamide, DMSO refers to dimethyl sulfoxide, TEA refers to triethylamine, TFA refers to trifluoroacetic acid, and THF refers to tetrahydrofuran.

### Methods of Preparation

Compounds of the general formula I wherein L=1; n=1; M=CH═CH and X═O may be prepared as outlined in Scheme 1. N-Boc-(d,l)-allylglycine (1) may be converted to the bromomethyl ketone 2 by treatment with iso-butylchloroformate followed by treatment of the intermediate mixed anhydride with diazomethane to provide the diazomethyl ketone (not shown). Treatment of the diazomethyl ketone with 30% HBr/HOAc provides the bromothylketone 2. Conversion of the bromomethylketone 2 to the hydroxymethylketone 3 may be effected by treatment of 2 with benzoylformic acid followed by saponification of the intermediate formate ester with an aqueous base such as potassium hydrogen carbonate. Etherification of alcohol 3 may be effected by treatment with allyliodide and silver oxide to provide the ether 4. Reduction of 4 with a reducing agent common to the art such as sodium borohydride followed by protection with dimethoxypropane provides the diene 5. The diene 5 may be cyclised to the 3,4,5,8-tetrahydro-2H-oxocin 6 by treatment with an olefin metathesis catalyst such as bis(tricyclohexylphosphine)benzylidine ruthenium(IV)dichloride. Removal of the acid labile protecting groups may be effected with and acid such as HCl or TFA to provide the amino alcohol 7. Amino alcohol 7 may be acylated with an acid such as N-Boc-leucine in the presence of a coupling agent common to the art such as EDC. Removal of the N-Boc protecting group may be effected with an acid such as HCl or TFA to provide the amine salt 8. The amine salt 8 may be acylated with an acid such as 2-naphthoic acid in the presence of a coupling agent such as EDC. The resulting alcohol (not shown) may be oxidized to the ketone 9 with an oxidizing agent common to the art such as Dess-Martin periodoindane or Swern conditions. These procedures may also be utilized with chiral N-Boc-L-allylglycine as starting material to provide a single diasteromer.

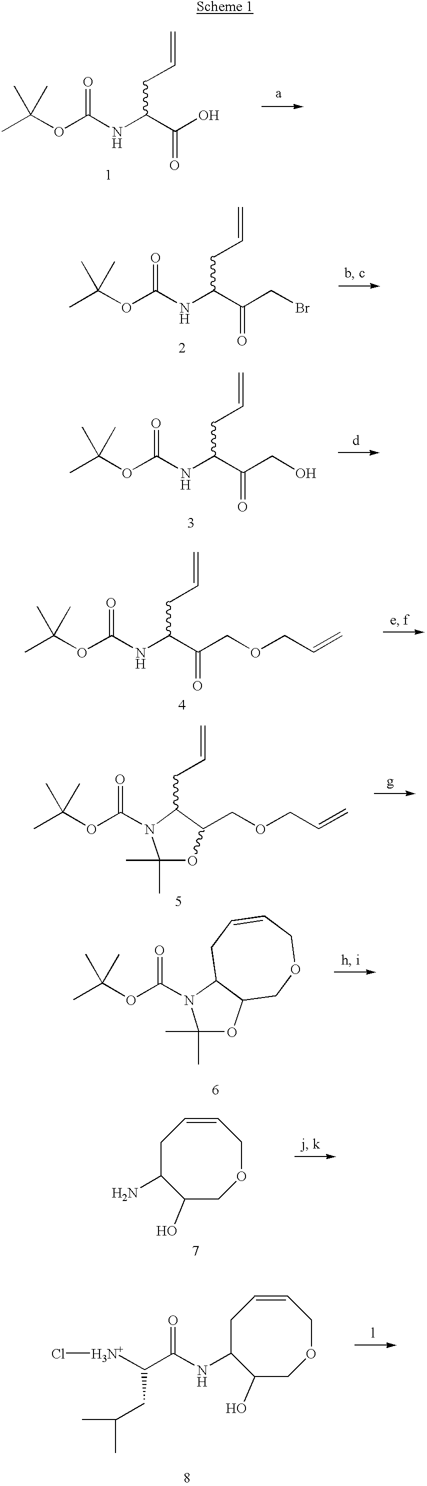

Alternatively, the saturated derivatives may be prepared as outlined in Scheme 2. Coupling of amino alcohol 10 with Cbz-leucine in the presence of a coupling agent such as EDC provides the amide 11. Reduction of the olefin and concomitant removal of the Cbz protecting group with 10% Pd on carbon in the presence of hyrogen gas gives 12. Coupling followed by oxidation with an oxidant such as Dess-Martin periodinane provides the ketone 13.

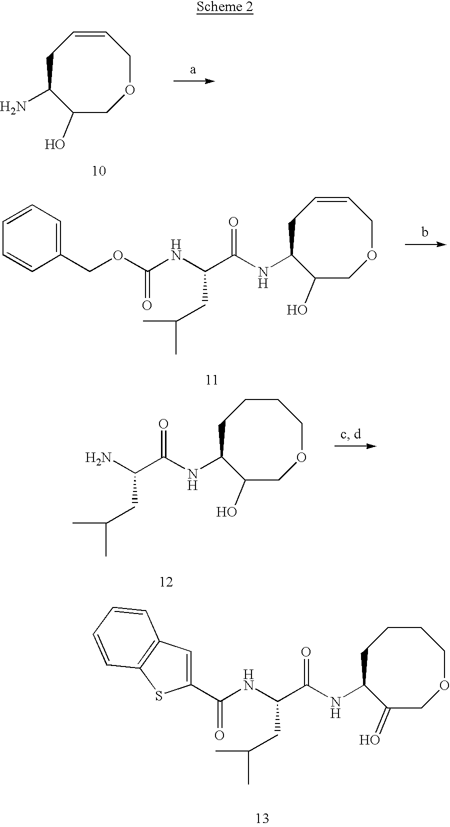

Reagants and Conditions

a.) CBz-leucine, EDC; b.) 10% Pd/C, H₂; c.) benzofuran-2-carboxylic acid, EDC; d.) Dess-Martin periodinane.

Compounds of the general formula I wherein L=0; n=1; M=CH₂CH₂ and X═O may be prepared as outlined in Scheme 3. Evans boronate aldol reaction of the chiral imide 14 with allyloxy acetaldehde provides 15. Removal of the chiral auxiliary under standard conditions provides the intermediate carboxylic acid (not shown) which was treated with diphenylphosphoryl azide to provide the cylic carbamate 16. Ring closing olefin metathesis of 16 with bis(tricyclohexylphosphine)benzylidine ruthenium(IV)dichloride provides the intermediate oxepin (not shown) which is reduced with hydrogen in the presence of 10% Pd/C to provide the oxepane 17. Saponification of the carbamate may be effected by treatment of 17 with lithium hydroxide followed by treatment with di-tert-butyldicarbonate to provide the tert-butyl carbamate (not shown). Removal of the tert-butoxycarbonyl protecting group under standard acidic conditions provided the amino alcohol derivative 18. Coupling of amino alcohol 18 with N-Boc-leucine may be effected by coupling reagents common in the art such as EDC. Removal of the tert-butoxycarbonyl protecting group under standard acidic conditions provided 19. Coupling of 19 with benzo[b]thiophene-2-carboxylic acid in the presence of a coupling agent such as EDC followed by oxidation with an oxidizing agent such as Dess-Martin periodinane provides the ketone derivative 20.

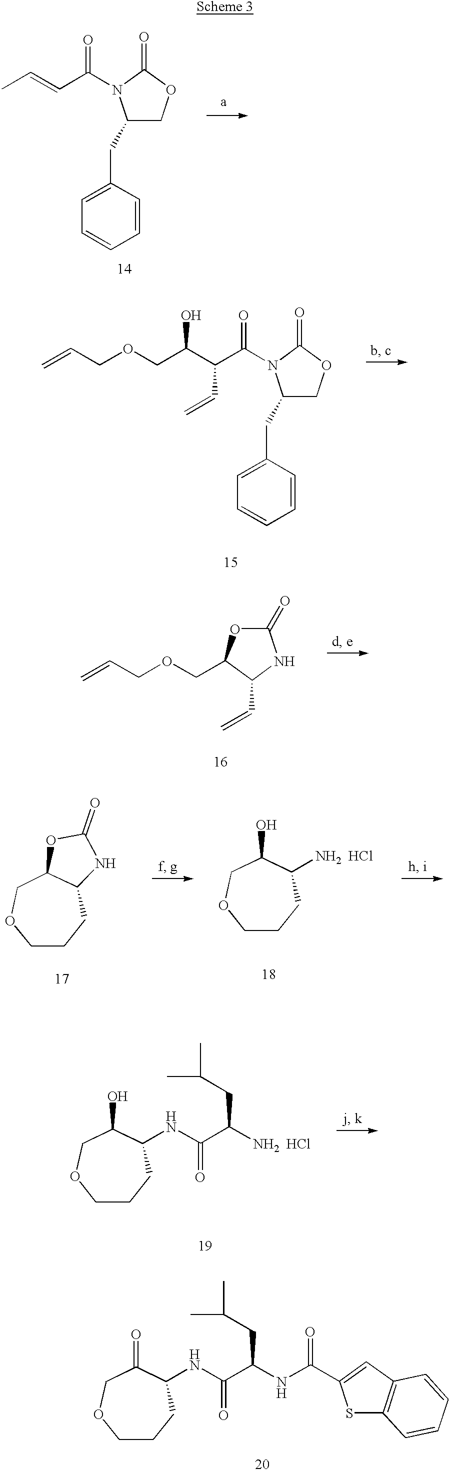

Reagents and Conditions

a.) n-Bu₂BOTf, TEA H₂C═CCH₂OCH₂CHO, −78° C. to 0° C.; b.) LIOH, H₂O₂, THF:H₂O; c.) (PhO)₂P(O)N₃, TEA, toluene, 120° C.; d.) bis(tricyclohexylphosphine)benzylidine ruthenium(IV) dichloride, CH₂Cl₂, reflux; e.) 10% Pd/C, H₂, CH₃OH; f.) LiOH, H₂O, CH₃OH then Boc₂O, NaOH, dioxane; g.) 4N HCl in dioxane; h.) N-Boc-leucine, EDC, CH₂Cl₂; i.) 4N HCl in dioxane; j.) benzo[b]thiophene-2-carboxylic acid, EDC, HOBt; k.) Dess-Martin periodinane, CH₂Cl_{2.}

### Novel Intermediates

Referring to the methods of preparing the compounds of Formula I set forth in Schemes 1 above, the skilled artisan will appreciate that the present invention includes all novel intermediates required to make the compounds of Formula I. Specifically, the present invention includes the following novel intermediates:

2,2-Dimethyl-3a,6,9,9a-4H-3,5dioxa-1-azacyclopentacyclooctene-1-carboxylic acid tert-butyl ester;

(S)-2,2-Dimethyl-3a,6,9,9a-4H-3,5dioxa-1-azacyclopentacyclooctene-1-carboxylic acid tert-butyl ester;

4-Amino-3,4,5,8-tetrahydro-2H-oxocin-3-ol;

(S)-4-Amino-3,4,5,8-tetrahydro-2H-oxocin-3-ol;

[(S)-1-(3-Hydroxy-3,4,5,8-tetrahydro-2H-oxocin-4-ylcarbamoyl)-3-butyl]carbamic acid tert-butyl ester;

[(S)-1-((S)-3-Hydroxy-3,4,5,8-tetrahydro-2H-oxocin-4-ylcarbamoyl)-3-butyl]carbamic acid tert-butyl ester; and

(S)-2-Amino-4-methyl-pentanoic acid (3-hydroxy-3,4,5,8-tetrahydro-2H-oxocin-4-yl) amide;

(S)-2-Amino-4-methyl-pentanoic acid ((S)-3-hydroxy-oxocan-4-yl) amide;

(3S, 4R)-4-Amino-oxepan-3-ol; and

(S)-2-Amino-4-methyl-pentanoic acid ((3S, 4R)-3-hydroxy-oxepan-4-yl) amide.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984. are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insulation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly as inhibitors of cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof an inhibitor of cathepsin K, including a compound of the present invention. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, , and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

### Biological Assays

The compounds of this invention may be tested in one of several biological assays to determine the concentration of compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 μM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

ν=VₘA/[Kₐ(l+I/K_{i,app})+A] (1)

where ν is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=ν_{SS}t+(ν₀−νₛₛ)[1−exp(−k_{obs}t)]/k_{obs} (2)

where [AMC] is the concentration of product formed over time t, ν₀ is the initial reaction velocity and νₛₛ is the final steady state rate. Values for k_{obs} were then analyzed as a linear function of inhibitor concentration to generate an apparent second order rate constant (k_{obs}/inhibitor concentration or k_{obs}/ [I]) describing the time-dependent inhibition. A complete discussion of this kinetic treatment has been fully described (Morrison et al., Adv. Enzymol. Relat. Areas Mol. Biol., 1988, 61, 201).

Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPMI-1640 medium, and incubated for 30 min on ice The cell suspension was mixed frequently.

The cells were washed ×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 min on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated ×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7g/litre of sodium bicarbonate. 3 mL aliquots of the cell suspension ( per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM 1 diluted to 100 ug/mL) and an isotype control (IgG2a diluted to 100 ug/mL). The tubes were incubate at 37° C. for 30 min. 0.5 mL aliquots of the cells were seeded onto sterile dentine slices in a 48-well plate and incubated at 37° C. for 2 h. Each treatment was screened in quadruplicate. The slices were washed in six changes of warm PBS (10 mL/well in a 6-well plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer / fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

### General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### EXAMPLES

In the following synthetic example, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### EXAMPLE 1

Preparation of Naphthylene-2-carboxylic acid [(S)-3-methyl-1-(3-oxo-3,4,5,8-tetrahydro-2H-oxocin-4-ylcarbamoyl)butyl]amide

a.) [1-(2-bromo-acetyl)but-3-enyl]-carbamic Acid Tert-butyl Ester

Isobutyl chloroformate (6.87 ml, 52.96 mmol) was added to a solution of N-Boc-D,L-allyl-glycine (9.5 g, 44.1 mmol) in N-methyl morpholine (5.82 ml, 52.9 mmol) in THF (200 mL) at −40° C. The reaction was stirred for 15 minutes whereupon it was filtered to remove the salts. The filtrate was added to a solution of diazomethane in Et₂O (300 ml) (generated from 1-methyl-3-nitro-1-nitrosoguanidine (10 g), 40% KOH (50 ml) and Et₂O (300 ml). The reaction mixture was stirred until complete by TLC analysis (ca. 30 mins.). 30% HBr in AcOH (15 mL) was added dropwise at -40° C. The reaction mixture was stirred at this temperature until complete as consumption of the starting material was observed (TLC). The reaction mixture was then diluted with ether, washed with water, aq. sat. NaHCO , brine, dried (magnesium sulfate), filtered, concentrated in vacuo to provide 8.47 g of [1-(2-bromo-acetyl)but-3-enyl]-carbamic acid tert-butyl ester. This material was used in the next reaction without further purification.

b.) [1-(2-hydroxy-acetyl)but-3-enyl]-carbamic Acid Tert-butyl Ester

To a solution of [1-(2-bromo-acetyl)but-3-enyl]-carbamic acid tert-butyl ester of Example 1a (8.5 g, 29.10 mmol) in DMF was added KF (2.53 g, mmol) followed by benzoylformic acid (5.24 g, 34.9 mmol). The reaction was stirred until complete consumption of the starting material was observed by TLC analysis (ca. 2 hours). The reaction was diluted with ether and washed successively with sat. K₂CO₃, water and brine. The organic layer was dried (MgSO₄), filtered and concentrated. The residue was dissolved in 1:1 THF:sat. KHCO₃ and stirred vigorously overnight whereupon the reaction was worked up to give the title compound as an oil: MS (EI) 230.2 (M+H⁺).

c.) [1-(2-allyoxy-acetyl)but-3-enyl]-carbamic Acid Tert-butyl Ester

To a solution of [1-(2-hydroxy-acetyl)but-3-enyl]-carbamic acid tert-butyl ester of Example 1b (2.0 g, 8.72 mmol) in CH₂Cl₂(50 mL) was added allyiodide (1.51 mL, 17.44 mmol) and silver(II)oxide (2.42 g, 10.46 mmol). The reaction was stirred at reflux overnight whereupon it was filtered and concentrated. Column chromatography (4:1 hexanes:ethyl acetate) of the residue provided 0.47 g of the title compound: 1H NMR (400 MHz) d 5.85 (m, 1H), 5.63 (m, 1H), 5.3-5.4 (m, 4H), 4.6 (m, 1H), 4.16 (m, 2H), 4.04 (m, 2H), 2.57 (m, 1H), 2.36 (m, 1H), 1.40 (s, 9H).

d.) 1-(2-allyoxy-1-hydroxy-ethyl)but-3-enyl]-carbamic Acid Tert-butyl Ester

To a solution of [1-(2-allyoxy-acetyl)but-3-enyl]-carbamic acid tert-butyl ester of Example 1c (0.47 g) in methanol (10 mL) was added sodium borohydride (100 mg) the reaction was stirred for ca. 30 mins. whereupon it was concentrated. The residue was dissolved in ethyl acetate and washed with 1N HCl, sat NaHCO₃, water, brine, dried (MgSO₄), filtered and concentrated to give 472 mg of the title compound: MS(EI) 272.3 (M+H⁺).

e.) 4-allyl-5-allyloxymethyl-2,2-dimethyl-oxazolidine-3-carboxylic Acid Tert-butyl Ester

To a solution of 1-(2-allyoxy-1-hydroxy-ethyl)but-3-enyl]-carbamic acid tert-butyl ester of Example 1d (472 mg, 1.74 mmol) in dichloromethane (10 mL was added 2,2-dimethoxypropane (2.14 mL, 17.4 mmol) and catalytic CSA. The reaction was stirred at reflux overnight whereupon it was concentrated and chromatographed (4:1 hexanes:ethyl acetate) to provide 486 mg of the title compound as an oil: MS(EI) 312.3 (M+H⁺).

f.) 2,2-dimethyl-3a,6,9,9a-4H-3,5dioxa-1-azacyclopentacyclooctene-1-carboxylic Acid tert-butyl ester

To a solution of 4-Allyl-5-allyloxymethyl-2,2-dimethyl-oxazolidine-3-carboxylic acid tert-butyl ester of Example 1e (486 mg, 1.56 mmol) in toluene (78 mL) was added bis(tricyclohexylphosphine)benzylidine ruthenium(IV)dichloride (128 mg). The reaction was heated to 80° C. for ca. 3 hours whereupon it was concentrated and the residue was chromatographed (6:1 hexanes: ethyl acetate) to give 262 mg of the title compound: MS (EI) 284.3 (M+H⁺).

g.) 4-Amino-3,4,5,8-tetrahydro-2H-oxocin-3-ol

To a solution of 2,2-Dimethyl-3a,6,9,9a-4H-3,5dioxa-1-azacyclopentacyclooctene-1-carboxylic acid tert -butyl ester of Example 1f (160 mg, 0.56 mmol) in CH₂Cl₂ (5.0 mL) was added TFA (0.5 mL). The mixture was maintained at −20° C. overnight whereupon it was concentrated diluted with ethyl acetate and washed with 40% KOH, brine, dried (Na₂SO₄) filtered and concentrated. The residue was then dissolved in THF:H₂O: TFA (2: 1:1) and stirred overnight at room temperature. The reaction was concentrated and worked-up as before to give 140 mg of the title compound: MS(EI) 144 (M+H⁺).

h.) [(S)-1-(3-hydroxy-3,4,5,8-tetrahydro-2H-oxocin-4-ylcarbamoyl)-3-butyl]carbamic Acid Tert-butyl Ester

To a solution of 4-Amino-3,4,5,8-tetrahydro-2H-oxocin-3-ol of Example 1g (140 mg, 0.54 mmol) in CH₂Cl₂ (10 mL) was added NMM (0.3 mL, 2.72 mmol), EDC (108 mg, 0.57 mmol) and N-Boc-leucine (132 mg, 0.57 mmol). The reaction was stirred at room temperature for 20 mins. whereupon it was concentrated. The residue was dissolved in ethyl acetate and washed with 1N HCl, sat. NaHCO₃, brine, dried (MgSO₄) filtered and concentrated. Column chromatography of the residue (3:1 ethyl acetate:hexanes) provided 60 mg of the title compound: MS(EI) 357.4 (M+H⁺).

i.) (S)-2-Amino-4-methyl-pentanoic acid (3-hydroxy-3,4,5,8-tetrahydro-2H-oxocin-4-yl) Amide

To a solution of [(S)-1-(3-Hydroxy-3,4,5,8-tetrahydro-2H-oxocin-4-ylcarbamoyl)-3-butyl]carbamic acid tert-butyl ester of Example 1h (60 mg) in CH₂Cl₂ (3.0 mL) was added TFA (0.5 mL). The reaction was stirred at room temperature until complete by TLC analysis whereupon it was concentrated to give the title compound: MS(EI) 257.3 (M+H⁺).

j.) Naphthylene-2-carboxylic Acid[(S)-3-methyl-1-(3-hydroxy-3,4,5,8-tetrahydro-2H-oxocin-4-ylcarbamoyl)-butyl]-amide

To a solution of (S)-2-Amino-4-methyl-pentanoic acid (3-hydroxy-3,4,5,8-tetrahydro-2H-oxocin-4-yl) amide of Example 11 (62 mg, 0.17 mmol) in CH₂Cl₂ (5.0 mL) was added N-methylmorpholine (0.09 mL, 0.84 mmol), EDC (38.7 mg, 0.20 mmol) and 2-naphthoic acid (34.7 mg, 0.2 mmol). The reaction was stirred at room temperature until complete as determined by TLC analysis. The reaction was worked up and chromatographed (3:1 ethyl acetate:hexanes) to give the title compound: MS(EI) 411.4 (M+H⁺).

k.) Naphthylene-2-carboxylic acid[(S)-3-methyl-1-(3-oxo-3,4,5,8-tetrahydro-2H-oxocin-4-ylcarbamoyl)-butyl]-amide

To a solution of naphthylene-2-carboxylic acid[(S)-3-methyl-1-(3-hydroxy-3,4,5,8-tetrahydro-2H-oxocin-4-ylcarbamoyl)-butyl]-amide of Example 1j (9.5 mg, 0.02 mmol) in CH₂Cl₂ (2.0 mL) was added Dess-Martin periodoindane (15 mg). The reaction was stirred at room temperature for ca. 1 hour whereupon it was diluted with CH₂Cl₂ and Na₂S₂₃ solution and NaHCO₃ solutions were added. The organic layer was washed with brine, dried (MgSO₄), filtered and concentrated. Column chromatography of the residue (3% methanol:dichloromethane) gave 3.9 mg of the title compound: MS(EI) 409.4 (M+H⁺).

### EXAMPLE 2

### Benzo[b]thiophene-2-carboxylic acid [(S)-3-methyl-1-((S)-3-oxo-3,4,5,8-tetrahydro-2H-oxocin-4-ylcarbamoyl)-butyl]-amide

a.) (S)-2-Amino-4-methyl-pentanoic acid ((S)-3-hydroxy-3,4,5,8-tetrahydro-2H-oxocin-4-yl) amide

Following the procedures of Example 1a-i except substituting N-Boc-L-allylglycine for N-Boc-(D, L)-allylglycine of Example la, the title compound was prepared.

b.) Benzo[b]thiophene-2-carboxylic acid [(S)-3-methyl-1-(3-hydroxy-3,4,5,8-tetrahydro-2H-oxocin-4-ylcarbamoyl)-butyl]-amide

To a solution of the the compound of Example 2a (50mg) in DMF was added benzo[b]thiophene-2-carboxylic acid (33mg, 0.1 85mmol), followed by the addition of HOBT (4mg, 0.029mmol), EDC (36mg, 0.187mmol) and Et₃N (1 mL, 6.68mmol). The reaction was allowed to stir for 16h at RT and was then diluted with EtOAc, washed with a solution of saturated NaHCO₃ and brine. The organic layer was dried over NaSO₄, filtered and concentrated. The crude mixture was purified on silica gel column to yield 63 mg (81%) of the title compound: MS (El) 438.9 (M +Na).

c.) Benzo[b]thiophene-2-carboxylic acid f(S)-3-methyl-1-[(S)-3-oxo-3,4,5,8-tetrahydro-2H-oxocin-4-ylcarbamoyl)-butyl]-amide To a soultion of the compound of Example 2b (13 mg) dissolved in CH₂,Cl₂ was added Dess-Martin reagent (26mg, 0.061 mmol) at RT and stirred for 1h. The reaction mixture was diluted with CH₂Cl₂ and washed with 10% Na₂S₂O₃, saturated NaHCO₃ and brine. The organic layer was dried over Na₂SO₄, filtered and concentrated to yield 13mg (100%) of the title compound: ¹H NMR (400 MHz, CDCl₃), δ: 7.8 (m, 3H), 7.4 (m, IH), 7 (t, 1H), 5.8 (q, 1H), 5.5 (q, 1H), 1.0 (d, 6H), 1.0 (d, 6H). MS (EI) 436.9 (M +Na).

### EXAMPLE 3

### Preparation of 3-Methylbenzofuran-2-carboxylic Acid [(S)-3-methyl-1-((S)-3-oxo-3,4,5,8-tetrahydro-2H-oxocin-4-ylcarbamoyl)-butyl]-amide

Following the procedures of Example 2b-c except substituting 3-methylbenzofuran-2-carboxylic acid for benzo[b]thiophene-2-carboxylic acid, the title compound was prepared: ¹H NMR (400 MHz, CDCl₃), δ: 7.3-7.6 (m, 4H), 6.9-7.1 (2d, 2H), 5.8 (q, 1H), 5.5 (q, 1H), 1.0 (d, 6H), MS (EI) 412.97 (M +H), 434.93 (M +Na).

### EXAMPLE 4

### Preparation of Quinoxaline-2-carboxylic acid [(S)-3-methyl-1-((S)-3-oxo-3,4,5,8-tetrahydro-2H-oxocin-4-ylcarbamoyl)-butyl]-amide

Following the procedures of Example 2b-c except substituting quinoxaline-2-carboxylic acid for benzo[b]thiophene-2-carboxylic acid, the title compound was prepared: ¹H NMR (400 MHz, CDCl₃), δ: 9.5 (d, 1H), 8.3 (d, 1H), 8.0 (d, 2H), 7.7 (m, 3H), 5.6 (q, 1H), 5.4 (q, 1H), 1.0 (d, 6H). MS (EI) 410.99 (M +H), 432.95 (M +Na).

### EXAMPLE 5

### Preparation of Benzofuran-2-carboxylic acid [(S)-3-methyl-1-((S)-3-oxo-3,4,5,8-tetrahydro-2H-oxocin-4-ylcarbamoyl)-butyl]-amide

Following the procedures of Example 2b-c except substituting benzofuran-2-carboxylic acid for benzo[b]thiophene-2-carboxylic acid, the title compound was prepared: ¹H NMR (400 MHz, CDCI,), δ: 7.6 (d, 1H), 7.3-7.5 (m, 4H), 7.1 (d, 1H), 6.9 (d, 1H), 6.8 (q, 1H), 5.5 (q, 1H), 1.0 (d, 6H). MS (EI) 422.93 (M +Na).

### EXAMPLE 6

### Preparation of Benzo[b]thiophene-2-carboxylic acid [(S)-3-methyl-1-((S)-3-oxo-oxocan-4-ylcarbamoyl)-butyl]-amide

a.) [(S)-I ((S)-3-Hydroxy-3,4,5,8-tetrahydro-2H-oxocin-4-ylcarbamoyl-3-methyl-butyl]-carbamic acid benzyl ester

To the a solution of the compound of Example 2a (300mg, 1.67mmol) dissolved in DMF was added Cbz-leu-OH (486mg, 1.83mmol), followed by the addition of HOBT (42mg, 0.31mmol), EDC (350mg, 2.6mmol) and Et₃N (I mL, 6.68mmol). The reaction was allowed to stir for 16h at RT and was then diluted with EtOAc, washed with a solution of saturated NaHCO₃ and brine. The organic layer was dried over Na₂SO₄, filtered and concentrated. The crude mixture was purified on silica gel column to yield 360 mg (55%) of the title compound: ¹H NMR (400 MHz, CDCl₃), δ: 7.3-7.4 (m, 7H), 6.7 (d, 1H), 5.7 (q, 1H), 5.4 (d, 1H), 3.5-4.5 (m, 7H), 1.0 (d, 6H). MS (EI) 413.03 (M +Na).

b.) (S)-2-Amino-4-methyl-pentanoic acid ((S)-3-hydroxy-oxocan-4-yl)-amide

To a solution of the compound of Example 6a in EtOH (10mL) was added 10% Pd/C (180mg). The reaction was stirred under hydrogen atmosphere for 3h. The reaction mixture was filtered through celite bed and the filtrate concentrated to give 248mg (100%) of the title compound. MS (EI) 259.07 (M +H).

c.) Benzofuran-2-carboxylic acid [(S)-3-methyl-1-((S)-3-hydroxy-3,4,5,8-tetrahydro-2H-oxocin-4-ylcarbamoyl)-butyl]-amide

To a solution of the amine of Example 6b (20mg, 0.07mmol) dissolved in DMF was added benzo[b]thiophene-2-carboxylic acid (0.015 mg, 0.084mmol), followed by HOBT (2mg, 0.015mmol), EDC and (16mg, 0.083mmol). The reaction was allowed to stir for 16h at RT and was then diluted with EtOAc, washed with a solution of saturated NaHCO, and brine. The organic layer was dried over Na₂SO₄, filtered and concentrated. The crude mixture was purified on silica gel column to yield 25mg (79%) of the title compound. ¹H NMR (400 MHz, CDCI), δ: 7.8 (m, 3H), 7.5 (m, 2H), 6.6-6.8 (d, 2H), 5.1 (m, 1H), 4.7 (q, 1H), 3.5-3.9 (m, 3H), 1.5-1.8 (m, 7H), 1.0 (d, 6H).

d.) Benzofuran-2-carboxylic acid [(S)-3-methyl-1-((S)-3-oxo-3,4,5,8-tetrahydro-2H-oxocin-4-ylcarbamoyl)-butyl]-amide

Following the procedure of Example 2c except substituting the compound of Example 6c, the title compound was prepared.

### EXAMPLE 7

### Preparation of 5-(4-Trifluoromethyl-phenyl)-furan-2-carboxylic acid [(S)-3-methyl-1-((S)-3-oxo-oxocan-4-ylcarbamoyl)-butyl]-amide

Following the procedures of Example 6c-d except substituting 5-(4-trifluoromethyl-phenyl)-furan-2-carboxylic acid for benzo[b]thiophene-2-carboxylic acid the title compound was prepared.

### EXAMPLE 8

### Preparation of 1-Methyl-1H-indole-2-carboxylic acid [(S)-3-methyl-1-((S)-3-oxo-oxocan-4-ylcarbamoyl)-butyl]-amide

Following the procedures of Example 6c-d except substituting 1-methyl-1H indole-2-carboxylic acid for benzo[b]thiophene-2-carboxylic acid the title compound was prepared: MS (EI) 436.11 (M +Na).

### EXAMPLE 9

### Preparation of 3-Methyl-benzofuran-2-carboxylic acid [(S)-3-methyl-1-((S)-3-oxo-oxocan-4-ylcarbamoyl)-butyl]-amide

Following the procedures of Example 6c-d except substituting 3-methyl-benzofuran-2-carboxylic acid for benzo[b]thiophene-2-carboxylic acid the title compound was prepared: MS (EI) 415.08 (M +H), 437.04 (M +Na).

### EXAMPLE 10

### Preparation of Quinoxaline-2-carboxylic acid [(S)-3-methyl-1-((S)-3-oxo-oxocan-4-ylcarbamoyl)-butyl]-amide

Following the procedures of Example 6c-d except substituting quinoxaline-2-carboxylic acid for benzo[b]thiophene-2-carboxylic acid the title compound was prepared: ¹H NMR (400 MHz, CDCl₃), δ: 9.0 (d, 2H), 8.6 (d, 2H), 7.0-8.1 (m, SH), 4.8 (q, 1H), 3.5-4.2 (m, 6H), 1.5-1.8 (m, 7H), 1.0 (d, 6H). MS (EI) 414.09 (M +H).

### EXAMPLE 11

### Preparation of 4-Methoxy-quinoline-2-carboxylic acid [(S)-3-methyl-1-((S)-3-oxo-oxocan-4-ylcarbamoyl)-butyl]-amide

Following the procedures of Example 6c-d except substituting 4-methoxy-quinoline-2-carboxylic acid for benzo[bjthiophene-2-carboxylic acid the title compound was prepared: MS (EI) 442.05 (M +H), 883.09 (2M +H).

### EXAMPLE 12

### Preparation of 5-Methyl-benzo[b]thiophene-2-carboxylic acid [(S)-3-methyl-1-((S)-3-oxo-oxocan-4-ylcarbamoyl)-butyl]-amide

Following the procedures of Example 6c-d except substituting 5-methyl-benzo[b]thiophene-2-carboxylic acid for benzo[b]thiophene-2-carboxylic acid the title compound was prepared: MS (EI) 403.01 (M +Na).

### EXAMPLE 13

### Preparation of Benzofuran-2-carboxylic acid [(S)-3-methyl-1-((S)-3-oxo-oxocan-4-ylcarbamoyl)-butyl]-amide

Following the procedures of Example 6c-d except substituting benzofuran-2-carboxylic acid for benzo[b]thiophene-2-carboxylic acid the title compound was prepared.

### EXAMPLE 14

### Preparation of 5,6-Dimethoxy-benzofuran-2-carboxylic acid [(S)-3-methyl-1-((S)-3-oxo-oxepan-4-ylcarbamoyl)-butyl]-amide

a.) (S)-3-[(S)-2-((S)-2-Allyloxy-1-hydroxy-ethyl)-but-3-enoyl]-4-benzyl-oxazolidin-2-one

To a 0° C. solution of (S)-3-((E)-but-2-enoyl)-4-(2-methyl-benzyl)-oxazolidin-2-one (9.0 g, 36.7 mmol) in CH₂Cl₂(150 mL) was added dibutylboron triflate (40.3 mL of a 1 M solution in CH₂Cl_{2,} 40.3 mmol) followed by triethylamine (7.2 mL, 51.4 mmol). The reaction was then cooled to -78° C. and freshly distilled allyloxy acetaldehyde (5.5 g, 55.0 mmol) was added in a rapid dropwise fashion. The reaction was maintained at −78° C. for an additional 30 minutes then warmed to 0° C. for 1 hour whereupon it was quenched by the addition of pH 7 buffer (50 mL) and methanol (150 mL). To this mixture was added a 2:1 methanol/30% H O solution (150 mL) at a rate that maintains the internal temperature below 10° C. The reaction is stirred at. 0° C. for an additional hour whereupon it was concentrated to approximately one third its original volume. The solution was then extracted with ether (3 x's). The combined organic layers were washed with sat. NaHCO, 1N HCl, brine, Dried (MgSO ), filtered and concentrated. Column chromatography (3:1 hexanes:ethyl acetate) of the residue provided 9.3 grams (73%) of the title compound as a clear colorless oil.

b.) (S)-2-((S)-2-Allyloxy-l-hydroxy-ethyl)-but-3-enoic acid

To a 0° C. solution of the aldol adduct of Example 13a (10.3 g, 29.8 mmol) in 3:1 THF:H₂O (150 mL) was added 30% H₂O₂ (10.5 mL) followed by LIOH (50 mL of a 0.3 M solution). The reaction was stirred for 30 minutes whereupon 2N sodium sulfite was added (70 mL). The volatile organics were removed under vacuum and the resulting basic solution was extracted with ethyl acetate (3×'s). The combined organic layers were washed with brine, dried (MgSO₄), filtered and concentrated to provide 4.9 grams (90%) of the title compound as a clear colorless oil which was of sufficient purity to use without purification.

c.) (4S,5S)-5-Allyloxymethyl-4-vinyl-oxazolidin-2-one

To a solution of the acid of Example 14b (0.2 g, 1.1 mmol) in toluene (10 mL) was added triethylamine (0.15 mL, 1.1 mmol) followed by diphenylphosphoryl azide (0.23 mL, 1.1 mmol. The reaction was stirred at 120° C. overnight whereupon it was concentrated. Column chromatography (1:1 hexane: ethyl acetate) of the resulting oil provided 0.13 g (65%) of the title compound as an oil.

d.) (3aS,8aS)-4,6,8a-Tetrahydro-1H-3,5-dioxa-] -azulen-2-one

To a solution of the diene of Example 14c (0.12 g, 0.65 mmol) in CH₂Cl₂ (10 mL) was added bis(tricyclohexylphosphine)benzylidine ruthenium(IV)dichloride (20 mg). The reaction was heated to reflux for approximately 90 minutes whereupon it was concentrated. Column chromatography (3:1 ethyl acetate:hexanes) of the residue provided 60 mg (60%) of the title compound as a brown solid.

e.) (3aS,8aS)-Hexahydro-3,5-dioxa-1-aza-azulen-2-one

To a solution of the olefin of Example 14d (0.6 g) in CH₃OH (20 mL) under nitrogen was added 10% Pd/C (150 mg). The mixture was evacuated and stirred under a balloon atmosphere of hydrogen overnight whereupon the mixture was filtered through a pad of celite with CH₂Cl₂ concentrated and chromatographed (3:1 ethyl acetate:hexanes) to provide 496 mg of the title compound as an off-white powder.

f.) (3S, 4R)-3-Hydroxy-oxepan-4-yl)-carbamic Acid Tert-butyl Ester

To a solution of the compound of Example 14e (100 mg) in methanol (2 mL) was added LiOH/H₂O (1.6 mL of a 2M solution) the reaction was stirred until complete consumption of the starting material was observed by TLC analysis whereupon the methanol was removed in vacuo. To the resulting liquid at 0° C. was added 1N NaOH (0.64 mL) and di-tert-butyl dicarbonate (0.16 mL). The reaction was warmed to room temperature and additional di-tert-butyl dicarbonate was added. After I hour the reaction was diluted with ethyl acetate and washed with water (3x's). The organic layer was dried, filtered and concentrated to provide 100 mg of the title compound.

g.) (3S, 4R)-4-amino-oxepan-3-ol

The compound of Example 14f (0.1 g) was suspended in 4M HCl/dioxane (1.1 mL) until complete consumption of the starting material. The reaction was concentrated and azeotroped with toluene to provide the title compound (0.075 g).

h.) [(R)-1-((3S, 4R)-3-Hydroxy-oxepan-4-ylcarbamoyl)-3-methyl-butyl]-carbamic Acid Tert-butyl Ester

Following the procedure of Example 1h except substituting the compound of Example 14g the title compound was prepared.

j.) (S)-2-Amino-4-methyl-pentanoic acid ((3S, 4R)-3-hydroxy-oxepan-4-yl) amide

Following the procedure of Example 14g except substituting the compound of Example 14h the title compound was prepared.

k.) 5,6-Dimethoxy-benzofuran-2-carboxylic acid [(S)-3-methyl-1-((S)-3-hydroxy-oxepan-4-ylcarbamoyl)-butyl]-amide

Following the procedure of Example 1h except substituting the compound of Example 14j and 5,6-dimethoxybenzofuran-2-carboxylic acid for N-Boc-leucine the title compound was prepared.

l.) 5,6-Dimethoxy-benzofuran-2-carboxylic acid [(S)-3-methyl-1-((S)-3-oxo-oxepan-4-ylcarbamoyl)-butyl]-amide

Following the procedure of Example 2c except substituting the compound of Example 14k the title compound was prepared.

### EXAMPLE 15

### Benzo[b]thiophene-2-carboxylic Acid [(S)-3-methyl-1-((S)-3-oxo-oxocan-4-ylcarbamoyl)-butyl]-amide

Following the procedures of Examples 14k-I except substituting benzo[b]thiophene-2-carboxylic acid for 5,6-dimethoxybenzofuran-2-carboxylic acid the title compound was prepared.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A compound of Formula I: 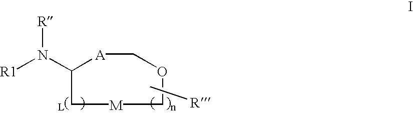
wherein:
A is C(O) or CH(OH);
R¹ is 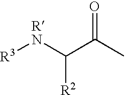
R¹ is selected from the group consisting of: H, C₁₋₆alkyl, Ar—C₀₋₆alkyl, or Het—C₀₋₆alkyl;
R″ is selected from the group consisting of: H, C₁₋₆alkyl, Ar—C₀₋₆alkyl, or Het-C₀₋₆alkyl;
R′″ is selected from the group consisting of: H, C₁₋₆alkyl, C₃₋₆cycloalkyl-C₀₋₆alkyl, Ar—C₀₋₆alkyl, or Het—C₀₋₆alkyl;
R² is selected from the group consisting of: H, C₂₋₆alkenyl, C₂₋₆alkynyl, Het, Ar or C₁₋₆alkyl optionally substituted by OR⁶, SR⁶, NR⁶₂, R⁶NC(O)OR⁵, CO₂R⁶, CO₂NR⁶₂, N(C═NH)NH₂, Het or Ar;
R³ is selected from the group consisting of: H, C₁₋₆alkyl, C₃₋₆cycloalkyl-C₀₋₆alkyl, Ar—C₀₋₆alkyl, Het—C₀₋₆alkyl, R⁴C(O)—, R⁴C(S)—, R⁴SO₂—, R⁴OC(O)—, R⁴R⁷NC(O)—, R⁴R⁷NC(S)—, R⁷HNCH(R⁷)C(O)—, or R⁴OC(O)NR⁷CH(R⁷)C(O)—;
R⁴ is selected from the group consisting of: C₁₋₆alkyl, C₃₋₆cycloalkyl-C₀₋₆alkyl, Ar—C₀₋₆alkyl or Het—C₀₋₆alkyl;
R⁵ is selected from the group consisting of: H, C₁₋₆alkyl, Ar—C₀₋₆alkyl, or Het—C₀₋₆alkyl;
R⁶ and R⁷ are selected from the group consisting of: H, C₁₋₆alkyl, Ar—C₀₋₆alkyl, or Het—C₀₋₆alkyl;
M is selected from the group consisting of: HC═CH, H₂C—CH₂; H(OR²)C—C(OR²)H; H(OR²)C—CH₂; H(NR²H)C—C(NR²H)H; H(OR²)C—C(NR²H)H; and H(NR²H)C—CH_{2;}
n is 1-7;
L is 0-1;
and pharmaceutically acceptable salts, hydrates and solvates thereof.

2. A compound according to claim 1 wherein A is C(O).

3. A compound according to claim 1 wherein R″ and R′″ are both H.

4. A compound according to claim 1 wherein:
A is C(O);
M is selected from the group consisting of: HC═CH and H₂C—CH₂;
n is 1
L is 0 or 1;and
R′, R″ and R′″ are independently H.

5. A compound according to claim 4 wherein:
R² is C₁₋₆alkyl, optionally substituted by OR⁶, SR⁶, NR⁶₂, R⁶NC(O)OR⁵, CO₂R⁶, CO₂NR⁶₂, N(C═NH)NH₂, Het and Ar;
R³ is R⁴C(O)—; and
R⁴ is selected from the group consisting of: Ar—C₀₋₆alkyl and Het—C₀₋₆alkyl.

6. A compound according to claim 5 wherein in R⁴:
Ar—C₀₋₆alkyl is naphthylenyl; and
Het—C₀₋₆alkyl is selected from the group consisting of:
benzo[b]thiophenyl;
3-methyl-benzofuranyl;
quinoxalinyl;
benzofuranyl;
benzo[b]thiophenyl;
5-(4-trifluoromethyl-phenyl)-furanyl;
1-methyl-1H-indolyl;
3-methyl-benzofuran;
4-methoxy-quinolinyl;
5-methyl-benzo[b]thiophenyl; and
5,6-dimethoxy-benzofuranyl.

7. A compound according to claim 6 wherein:
naphthylenyl is naphthylen-2-yl;
benzo[b]thiophenyl is benzo[b]thiophen-2-yl;
3-methyl-benzofuranyl is 3-methyl-benzofuran-2-yl;
quinoxalinyl is quinoxaline-2-yl;
benzofuranyl is benzofuran-2-yl;
benzo[b]thiophenyl is benzo[b]thiophene-2-yl;
5-(4-trifluoromethyl-phenyl)-furanyl is 5-(4-trifluoromethyl-phenyl)-furan-2-yl;
1-methyl-i H-indolyl is 1-methyl-1H-indol-2-yl;
3-methyl-benzofuranyl is 3-methyl-benzofuran-2-yl;
4-methoxy-quinolinyl is 4-methoxy-quinoline-2-yl;
5-methyl-benzo[b)thiophenyl is 5-methyl-benzo[b]thiophene-2-yl; and
5,6-dimethoxy-benzofuranyl is 5,6-dimethoxy-benzofuran-2-yl.

8. A compound of claim 1 selected from the group consisting of:
naphthylene-2-carboxylic acid[(S)-3-methyl-1-(3-oxo-3,4,5,8-tetrahydro-2H-oxocin-4-ylcarbamoyl)-butyl]-amide;
benzo[b]thiophene-2-carboxylic acid [(S)-3-methyl-1-((S)-3-oxo-3,4,5,8-tetrahydro-2H-oxocin-4-ylcarbamoyl)-butyl]-amide;
3-methyl-benzofuran-2-carboxylic acid [(S)-3-methyl-1-((S)-3-oxo-3,4,5,8-tetrahydro-2H-oxocin-4-ylcarbamoyl)-butyl]-amide;
quinoxaline-2-carboxylic acid [(S)-3-methyl-1-((S)-3-oxo-3,4,5,8-tetrahydro-2H-oxocin-4-ylcarbamoyl)-butyl]-amide;
benzofuran-2-carboxylic acid [(S)-3-methyl-1-((S)-3-oxo-3,4,5,8-tetrahydro-2H-oxocin-4-ylcarbamoyl)-butyl]-amide;
benzo[b]thiophene-2-carboxylic acid [(S)-3-methyl-1-((S)-3-oxo-oxocan-4-ylcarbamoyl)-butyl]-amide;
5-(4-trifluoromethyl-phenyl)-furan-2-carboxylic acid[(S)-3-methyl-1-((S)-3-oxo-oxocan-4-ylcarbamoyl)-butyl]-amide;
1-methyl-1H-indole-2-carboxylic acid f(S)-3-methyl-1-((S)-3-oxo-oxocan-4-ylcarbamoyl)-butyl]-amide;
3-methyl-benzofuran-2-carboxylic acid [(S)-3-methyl-1-((S)-3-oxo-oxocan-4-ylcarbamoyl)-butyl]-amide;
quinoxaline -2-carboxylic acid [(S)-3-methyl-1-((S)-3-oxo-oxocan-4-ylcarbamoyl)-butyl]-amide
4-methoxy-quinoline-2-carboxylic acid[(S)-3-methyl-1-((S)-3-oxo-oxocan-4-ylcarbamoyl)-butyl]-amide;
5-methyl-benzo[b]thiophene-2-carboxylic acid [(S)-3-methyl-1-((S)-3-oxo-oxocan-4-ylcarbamoyl)-butyl]-amide;
benzofuran-2-carboxylic acid [(S)-3-methyl-1-((S)-3-oxo-oxocan-4-ylcarbamoyl)-butyl]-amide;
5,6-dimethoxy-benzofuran-2-carboxylic acid [(S)-3-methyl-1-((S)-3-oxo-oxepan-4-ylcarbamoyl)-butyl]-amide; and
benzo[b]thiophene-2-carboxylic acid [(S)-3-methyl-1-((S)-3-oxo-oxocan-4-ylcarbamoyl)-butyl]-amide.

9. A pharmaceutical composition comprising a compound according to claim 1 and a pharmaceutically acceptable carrier, diluent or excipient.

10. A pharmaceutical composition comprising a compound according to claim 8 and a pharmaceutically acceptable carrier, diluent or excipient.

11. A method of inhibiting a protease selected from the group consisting of a cysteine protease and a serine protease, comprising administering to a patient in need thereof an effective amount of a compound according to claim 1.

12. A method of inhibiting a protease selected from the group consisting of a cysteine protease and a serine protease, comprising administering to a patient in need thereof an effective amount of a compound according to claim 8.

13. A method according to claim 11 wherein said protease is a cysteine protease.

14. A method according to claim 12 wherein said protease is a cysteine protease.

15. A method according to claim 13 wherein said cysteine protease is cathepsin K.

16. A method according to claim 14 wherein said cysteine protease is cathepsin K.

17. A method of treating a disease characterized by bone loss comprising inhibiting said bone loss by administering to a patient in need thereof an effective amount of a compound according to claim 1.

18. A method according to claim 17 wherein said disease is osteoporosis.

19. A method according to claim 17 wherein said disease is periodontitis.

20. A method according to claim 17 wherein said disease is gingivitis.

21. A method of treating a disease characterized by excessive cartilage or matrix degradation comprising inhibiting said excessive cartilage or matrix degradation by administering to a patient in need thereof an effective amount of a compound according to claim 1.

22. A method according to claim 21 wherein said disease is osteoarthritis.

23. A method according to claim 21 wherein said disease is rheumatoid arthritis.

24. A method of treating a disease characterized by bone loss comprising inhibiting said bone loss by administering to a patient in need thereof an effective amount of a compound according to claim 8.

25. A method according to claim 24 wherein said disease is osteoporosis.

26. A method according to claim 24 wherein said disease is periodontitis.

27. A method according to claim 24 wherein said disease is gingivitis.

28. A method of treating a disease characterized by excessive cartilage or matrix degradation comprising inhibiting said excessive cartilage or matrix degradation by administering to a patient in need thereof an effective amount of a compound according to claim 8.

29. A method according to claim 28 wherein said disease is osteoarthritis.

30. A method according to claim 28 wherein said disease is rheumatoid arthritis.

31. A compound selected from the group consisting of:
2,2-Dimethyl-3a,6,9,9a-4H-3,5dioxa-1-azacyclopentacyclooctene-1-carboxylic acid tert-butyl ester;
(S)-2,2-Dimethyl-3a,6,9,9a-4H-3,5dioxa-1-azacyclopentacyclooctene-1-carboxylic acid tert-butyl ester;
4-Amino-3,4,5,8-tetrahydro-2H-oxocin-3-ol;
(S)-4-Amino-3,4,5,8-tetrahydro-2H-oxocin-3-ol;
[(S)-1-(3-Hydroxy-3,4,5,8-tetrahydro-2H-oxocin-4-ylcarbamoyl)-3-butyl]carbamic acid tert-butyl ester;
[(S)-1-((S)-3-Hydroxy-3,4,5,8-tetrahydro-2H-oxocin-4-ylcarbamoyl)-3-butyl]carbamic acid tert-butyl ester; and
(S)-2-Amino-4-methyl-pentanoic acid (3-hydroxy-3,4,5,8-tetrahydro-2H-oxocin-4-yl) amide;
(S)-2-Amino-4-methyl-pentanoic acid ((S)-3-hydroxy-oxocan-4-yl) amide;
(3S, 4R)-4-Amino-oxepan-3-ol; and
(S)-2-Amino-4-methyl-pentanoic acid ((3S, 4R)-3-hydroxy-oxepan-4-yl) amide.

32. Use of a compound according to any one of claims 1 to 8 in the manufacture of a medicament for inhibiting a protease selected from the group consisting of a cysteine protease and a serine protease.

33. A use according to claim 32 wherein said protease is a cysteine protease.

34. A use according to claim 33 wherein said cysteine protease is cathepsin K.

35. Use of a compound according to any one of claims 1 to 8 in the manufacture of a medicament for use in treating a disease characterized by bone loss.

36. A use according to claim 35 wherein said disease is osteoporosis.

37. A use according to claim 35 wherein said disease is periodontitis.

38. A use according to claim 35 wherein said disease is gingivitis.

39. Use of a compound according to any one of claims 1 to 8 in the manufacture of a medicament for use in treating a disease characterized by excessive cartilage or matrix degradation.

40. A use according to claim 39 wherein said disease is osteoarthritis.

41. A use according to claim 39 wherein said disease is rheumatoid arthritis.
